(19) United States
(12) Patent Application Publication
(10) Pub. No.: US 2025/0213789 A1
(43) Pub. Date: Jul. 3, 2025

(54) INFUSION PUMP UPSTREAM OCCLUSION DETECTION WITH AIR-IN-LINE ASSISTANCE

(71) Applicant: CareFusion 303, Inc., San Diego, CA (US)

(72) Inventors: Richard S. WU, San Diego, CA (US); Jay DAVE, Carlsbad, CA (US)

(21) Appl. No.: 18/851,478

(22) PCT Filed: Mar. 29, 2022
(86) PCT No.: PCT/US22/22387

§ 371 Date: Sep. 26, 2024

(51) Int. Cl. A61M 5/168; A61M 5/142; A61M 5/36

(52) U.S. Cl. CPC A61M 5/16854; A61M 5/142; A61M 5/365; A61M 2205/18; A61M 2205/3331

(57) ABSTRACT

An infusion device determines, using a pressure sensor, a pressure decay inside an infusion line associated with an infusion of a fluid to a patient by an infusion device, whereby the pressure decay does not satisfy a default pressure threshold for triggering an occlusion alarm. The infusion device further detects, using an air sensor, air inside the infusion line in an amount that does not satisfy a default air-in-line threshold for triggering an air-in-line alarm. Then, before the pressure decay satisfies the default pressure threshold, the infusion device triggers the occlusion alarm based on the determined pressure decay and the detected amount of air.

### CROSS REFERENCE TO RELATED APPLICATION

This is a National Stage Application filed under 35 U.S.C. 371 based on International Patent Application No. PCT/US2022/22387, filed on Mar. 29, 2022, the disclosure of which is incorporated herein by reference in its entirety.

### BACKGROUND

A common concern associated with infusion pumps is the occlusion of the pump's fluid line. A “downstream” occlusion typically occurs due to a blockage between the pump and the patient. An occlusion between the intravenous (IV) medication bag and the pump is called an “upstream occlusion.” An upstream occlusion typically results from a blockage on the upstream side of the pump, preventing the IV medication from being delivered. An upstream occlusion may also result, for example, from a clinician forgetting to unclamp the line between the bag and the pump prior to starting the infusion. In some instances, an upstream occlusion may slow the flow of fluid to the patient. Depending on how long an upstream occlusion is left unattended, air may begin to enter the fluid line due to the pump pulling against the blockage. Thus, patient care may be threatened by increasing the risk of undernutrition, undermedication, and air embolism.

Accordingly, many infusion pumps currently include pressure sensors to facilitate detection of occlusion. When a sensor detects a significant drop of pressure in the fluid line, an implication may be that the pressure drop is due to an upstream occlusion and therefore the infusion pump triggers an upstream occlusion alarm. While this method of upstream occlusion detection is effective for infusion pumps operating at most flow rates, the method can be ineffective for some infusion pumps operating at slower flow rates. When an occlusion occurs at slower flow rates, the pressure in the fluid line decreases at a correspondingly slower rate. Accordingly, the time between the occurrence of an upstream occlusion and the detection of the upstream occlusion may be minutes or even hours. A faster way to detect upstream occlusion, especially for infusion pumps operating at lower flow rates, is therefore desirable.

### SUMMARY

According to various aspects of the subject technology, a method includes determining, using a pressure sensor, a pressure decay inside an infusion line associated with an infusion of a fluid to a patient by an infusion device, whereby the pressure decay does not satisfy a default pressure threshold for triggering an occlusion alarm; detecting, using an air sensor, air inside the infusion line in an amount that does not satisfy a default air-in-line threshold for triggering an air-in-line alarm; and triggering, before the pressure decay satisfies the default pressure threshold, the occlusion alarm based on the determined pressure decay and the detected amount of air.

According to various aspects of the subject technology, a system includes one or more occluder valves comprising a lower occluder valve, the lower occluder valve configured to, when activated, cause a compression of a fluid tubing loaded into the infusion pump, the compression fluidically isolating a downstream portion of the fluid tubing from an upstream portion of the fluid tubing when the lower occluder valve is operating under a normal operating condition; a lower pressure sensor downstream of the lower occluder valve; an air-in-line sensor; a pressure sensor; an occlusion alarm; and a processor configured to: determine, using a pressure sensor, a pressure decay inside an infusion line associated with an infusion of a fluid to a patient by an infusion device, whereby the pressure decay does not satisfy a default pressure threshold for triggering the occlusion alarm; detect, using the air-in-line sensor, air inside the infusion line in an amount that does not satisfy a default air-in-line threshold for triggering an air-in-line alarm; and trigger, before the pressure decay satisfies the default pressure threshold, the occlusion alarm based on the determined pressure decay and the detected amount of air.

According to various aspects of the subject technology, a non-transitory, computer-readable medium includes instructions that, when executed by one or more processors, cause the one or more processors to: determine, using a pressure sensor, a pressure decay inside an infusion line associated with an infusion of a fluid to a patient by an infusion device, whereby the pressure decay does not satisfy a default pressure threshold for triggering an occlusion alarm; detect, using an air sensor, air inside the infusion line in an amount that does not satisfy a default air-in-line threshold for triggering an air-in-line alarm; and trigger, before the pressure decay satisfies the default pressure threshold, the occlusion alarm based on the determined pressure decay and the detected amount of air.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

For a better understanding of the various described implementations, reference should be made to the Detailed Description below, in conjunction with the following drawings. Like reference numerals refer to corresponding parts throughout the figures and description.

FIG. 1 depicts an example infusion device, shown in its intended environment, according to various aspects of the subject technology.

FIG. 2 depicts an example infusion device in perspective view with a front door open, showing an upstream portion and a downstream portion of a fluid infusion line in operative engagement with the example infusion device, according to various aspects of the subject technology.

FIG. 3A depicts an example graph with example curves representative of pressure in an infusion line under normal and occluded conditions, as well as thresholds for detecting the same, according to various aspects of the subject technology.

FIG. 3B depicts an example graph with example curves representative of an amount of air present in an infusion line under normal and occluded conditions, as well as thresholds for detecting the same, according to various aspects of the subject technology.

FIG. 4 depicts a first example process for triggering an occlusion alarm, according to various aspects of the subject technology.

FIG. 5 depicts a second example process for triggering an occlusion alarm, according to various aspects of the subject technology.

FIG. 6 depicts a conceptual diagram illustrating an example electronic system for detecting an upstream occlusion, according to various aspects of the subject technology.

### DETAILED DESCRIPTION

Reference will now be made to implementations, examples of which are illustrated in the accompanying drawings. In the following description, numerous specific details are set forth, in order to provide an understanding of the various described implementations. However, it will be apparent to one of ordinary skill in the art that the various described implementations may be practiced without these specific details. In other instances, well-known methods, procedures, components, circuits, and networks have not been described in detail so as not to unnecessarily obscure aspects of the implementations.

Upstream occlusions at very low flow rates (e.g., at less than or equal to 1 mL/hr) have been known to result in a very long time-to-alarm (“TTA”) and, in some cases, result in an air-in-line alarm being triggered before the occlusion is detected. In traditional pumps, alarms may occur based on pressure or air bubbles, with pressure-based alarms often being synonymous with occlusions (e.g., an occlusion alarm may trigger based on pressure). The subject technology provides an occlusion alarm under conditions wherein neither pressure-nor air-based alarms would be triggered. Accordingly, the subject technology may alert clinicians of an occlusion in the system much earlier than traditional mechanisms.

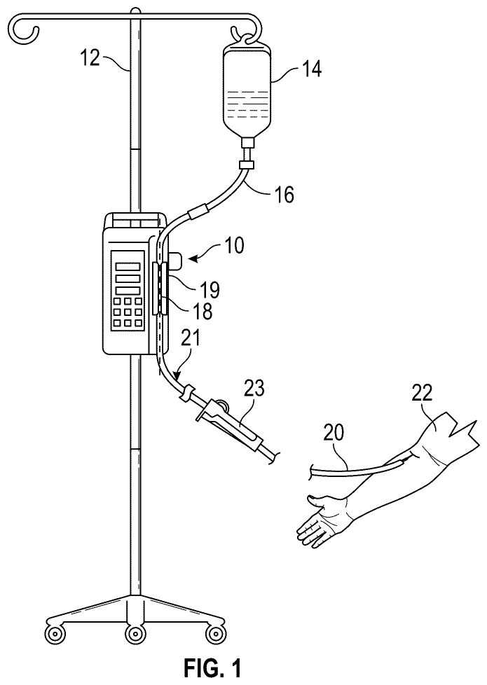

FIG. 1 depicts an example infusion device 10 (e.g., an infusion pump), shown in use in its intended environment, according to various aspects of the subject technology. In particular, the infusion device 10 is shown mounted to an intravenous (IV) pole 12 on which a fluid source 14 containing an IV fluid is held. The fluid source 14 is connected in fluid communication with an upstream portion 16 of a fluid infusion line 21. The fluid infusion line 21 is a conventional IV infusion type tube typically used in a hospital or medical environment and is made of any type of flexible tubing appropriate for use to infuse therapeutic fluids into a patient, such as silicone. A flexible portion 18 of the fluid infusion line 21 is mounted in operative engagement with a peristaltic pumping mechanism 19, for propelling fluid through a downstream portion 20 of the fluid infusion line 21, for example, to a patient's arm 22.

It will be understood by those skilled in the art that the upstream portion 16, the flexible portion 18, and the downstream portion 20 of the fluid infusion line 21 may be portions of the same continuous flexible tubing, with the portions defined by their position relative to the peristaltic pumping mechanism 19. For convenience, the continuous length of flexible tubing is indicated by numeral 21. A roller clamp 23 (e.g., configured to provide for mechanical compression of the line to block the flow) may be positioned on the downstream portion 20 of the fluid infusion line 21 between the infusion device 10 and the patient's arm 22. In this context, the term “upstream” refers to that portion of the flexible tubing that extends between the fluid source 14 and the peristaltic pumping mechanism 19, and the term “downstream” refers to that portion of the flexible tubing that extends from the peristaltic pumping mechanism 19 to the patient's arm 22.

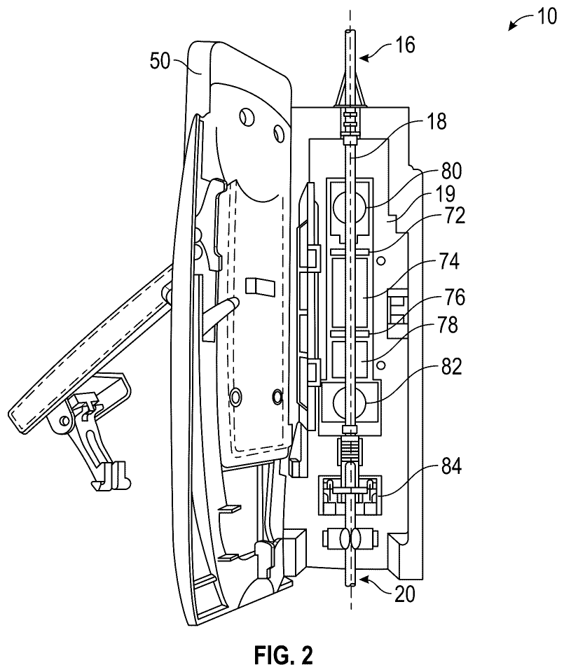

Turning now to FIG. 2, an infusion device 10 is shown in perspective view with a front door 50 open, showing an upstream portion 16 and a downstream portion 20 of fluid infusion line 21 in operative engagement with the infusion device 10. The infusion device 10, in operative engagement with a peristaltic pumping mechanism 19, directly acts on a flexible portion 18 of the fluid infusion line 21 that connects the upstream portion 16 to the downstream portion 20 of the fluid infusion line 21 to form a continuous fluid infusion line 21, extending from the respective fluid supply 14 of FIG. 1, to the patient's arm 22 of FIG. 1, through which fluid is acted upon by the pump to move fluid downstream to the patient's arm 22. Specifically, a peristaltic pumping mechanism 19 acts as the flow control device of the infusion device 10 to move fluid though the fluid infusion line 21. The upstream portion 16, the downstream portion 20, and/or the flexible portion 18 of the fluid infusion line 21 may be coupled to a pump cassette or a cartridge that is configured to be coupled to the infusion device 10, such as the type described in U.S. Pat. No. 10,226,571, which issued on Mar. 12, 2019, is currently owned by applicant, and is incorporated by reference herein.

The type of peristaltic pumping mechanism 19 may vary and may be, for example, a multi-finger pumping mechanism. For example, the pumping mechanism may be of the “four finger” type and include an upstream occluding finger 72, a primary pumping finger 74, a downstream occluding finger 76, and a secondary pumping finger 78. The “four finger” pumping mechanism, as well as mechanisms used in other linear peristaltic pumps, operate by sequentially pressing on a segment of the fluid conduit by means of the upstream occluding finger 72, primary pumping finger 74, downstream occluding finger 76, and secondary pumping finger 78. The pressure is applied in sequential locations of the fluid infusion line 21, beginning at the upstream end of the peristaltic pumping mechanism 19 and working toward the downstream end. At least one finger is always pressing hard enough to occlude the fluid infusion line 21. As a practical matter, one finger does not retract from occluding the tubing until the next one in sequence has already occluded the tubing; thus, at no time is there a direct fluid path from the fluid supply to the patient. The operation of peristaltic pumps including four finger pumps is well known to those skilled in the art and no further operational details are provided here.

In this particular implementation, FIG. 2 depicts a downstream pressure sensor 82 in the infusion device 10 at a downstream location with respect to the peristaltic pumping mechanism 19. The downstream pressure sensor 82 is mounted to the infusion device 10 and is located adjacent to the pumping mechanism 19. The downstream pressure sensor is also located downstream from the flow control device, that is, at a location between the pumping mechanism 19 and the patient's arm 22 of FIG. 1, so that the connection of the correct fluid supply with the correct pump may be verified before any fluid is pumped to the patient.

With reference still to FIG. 2, an upstream pressure sensor 80 is also depicted. The upstream pressure sensor is associated with the peristaltic pumping mechanism 19 and, in this example, is further provided as an integral part of the infusion device 10. It may be located adjacent and upstream in relation to the peristaltic pumping mechanism 19, for example, at a location between the fluid source 14 of FIG. 1 and the peristaltic pumping mechanism 19, so that the connection of the correct fluid supply with the correct pump may be verified before any fluid is pumped to the patient. For the purpose of this disclosure the term “pressure sensor” may also be used to also describe or include a force sensor. The pressure sensors described herein may be replaced by force sensors for all implementations described herein. A force sensor looks at compression force on the tubing, and a pressure sensor looks at the pressure of the tubing as it is compressed inside the pump. Both mechanisms are contemplated and the term “pressure sensor” (and the values read therefrom) as used herein should be interpreted to be either.

According to various implementations, the infusion device 10 may include one or more sensors for detecting air within the fluid infusion line 21. In the depicted example, a sensor 84 is configured within the infusion device 10, located beneath the flexible portion 18 of the fluid infusion line 21 and adjacent to the peristaltic pumping mechanism 19. While depicted as part of the infusion device 10 and downstream of the peristaltic pumping mechanism 19, sensor 84 may be located upstream of the peristaltic pumping mechanism 19 or may be separate and independent of the infusion device 10.

In some implementations, sensor 84 may include light or ultrasonic sensors. Accordingly, electromagnetic energy (e.g., light) or sound energy (e.g., an ultrasonic pulse) is passed through the fluid infusion line 21 (e.g., from a transducer in the door 50), and the sensor 84 monitors variations in the received energy. Because air generally transmits light and/or sound energy in a different fashion than do intravenous fluid solutions, due to different transmission properties such as absorption and/or refractivity, monitoring variations in the light's or sound's ability to pass through the solution can give a generally accurate determination that air exists in the fluid line.

An analog-to-digital converter may receive the output signals from the sensor 84 and convert them to a digital format at a particular sample rate controlled by a processor. The output signals indicate the amount of air in the line at a particular point in time. In some implementations, an age determiner, such as a volume accumulator, may provide an age value for the output digital signals, with the age value a function of the volume that has been introduced through the fluid line. (A clock could also provide an age value based upon the time that the output digital signal is generated or received, depending on the particular system.) The processor may receive the digital signals, process them, and determine an air concentration value representing air passing through the fluid delivery system. The air concentration value may represent a number of bubbles in the fluid infusion line 21, a volume of air in the fluid infusion line 21, or some combination of both. One or more alarms may be provided (e.g., integrated within the infusion device 10 or into a remote device) to indicate an unsatisfactory air concentration value in the fluid infusion line 21. In some implementations, the concentration may be assessed as an accumulation over a period of time or an accumulation of total amount of air detected by the device.

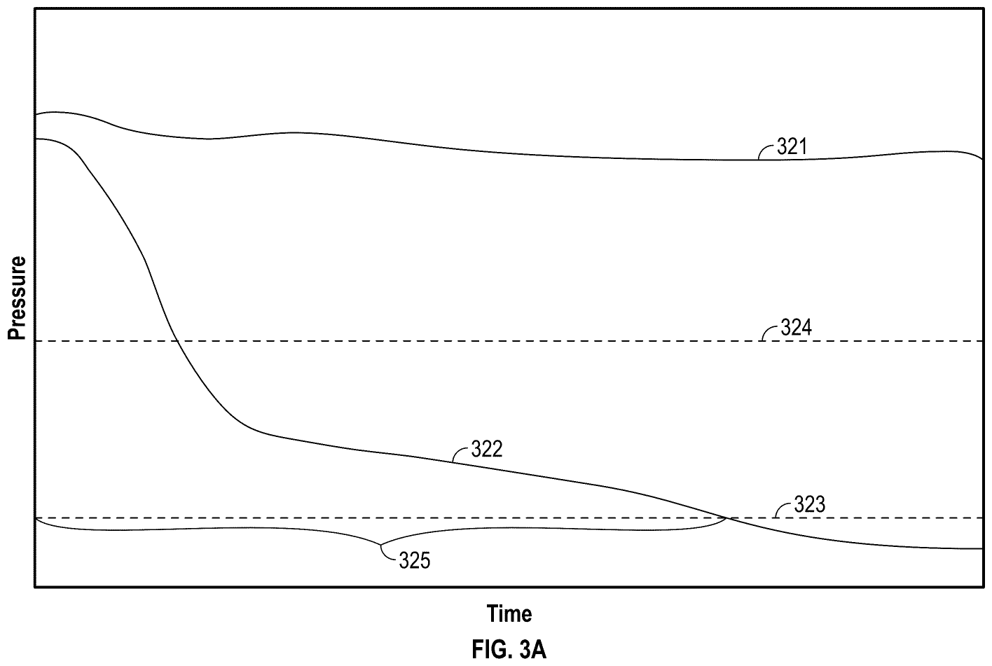

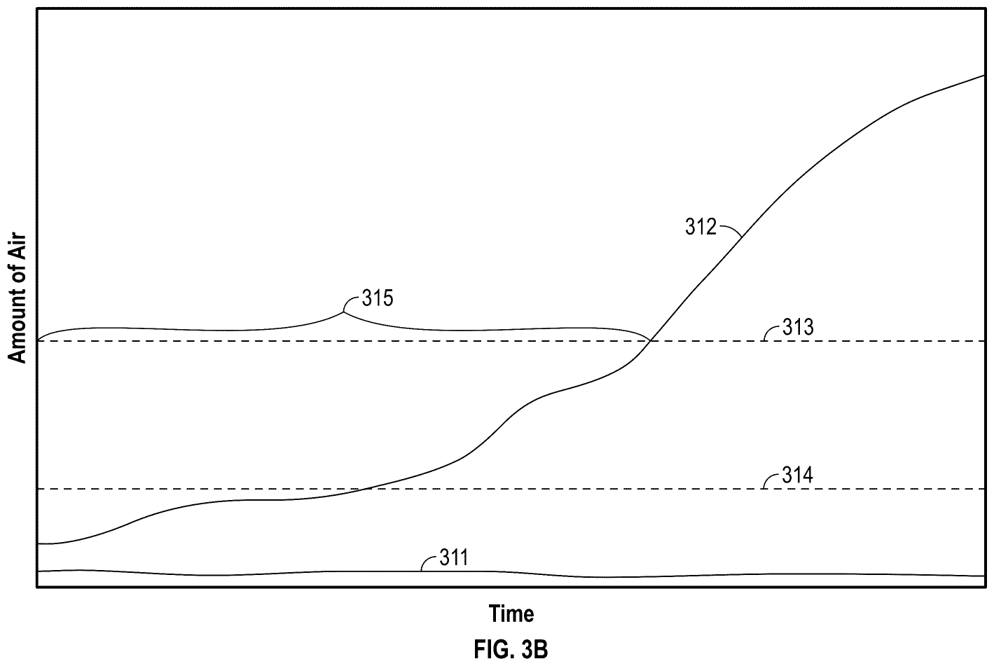

FIG. 3A depicts a graph with example curves representative of pressure in a fluid infusion line (e.g., the fluid infusion line 21 of FIG. 1) under normal and occluded conditions, including thresholds for detecting the same, according to various aspects of the subject technology. As described previously, a processor (e.g., within the infusion device 10 of FIGS. 1 and 2) may detect pressure within the fluid infusion line using a sensor (e.g., the upstream pressure sensor 80 of FIG. 2). The x-axis of the depicted graph represents time, and the y-axis represents pressure. Curve 321 represents the pressure in a fluid infusion line in the absence of an upstream occlusion. As depicted, the pressure remains stable in the absence of an occlusion. Curve 322, on the other hand, represents pressure during an upstream occlusion. As depicted, during an upstream occlusion, the pressure decreases over time.

Default pressure threshold 323 represents a default pressure threshold for triggering an upstream occlusion alarm based on pressure. For example, the default pressure threshold 323 may be pre-programmed into the infusion device 10 at the time the infusion is initiated. When there is an occlusion on the upstream side, the peristaltic pumping mechanism 19 of the infusion device will create a suction on the infusion line, causing the line to collapse and/or pinch. With the collapse of the tubing, the pressure sensor (e.g., the upstream pressure sensor 80 of FIG. 2) will measure a pressure drop and trigger an alarm once the pressure drops below the default pressure threshold 323. Generally, an upstream occlusion will be indicated by a fast drop in pressure. However, at lower flow rates (e.g., at or less than 1 mL/hr) when the pump is barely pumping fluid, the pressure drop will be much more gradual, resulting in a greater time to alarm time period 325. It has been found that, in some instances, detection may not occur for up to 2 hours. Therefore, under such conditions, it would be desirable to have the pump trigger an alarm faster.

Accordingly, the adjusted pressure threshold 324 represents an alternative pressure threshold that may be used instead of the default pressure threshold 323 under certain conditions. In some conditions, the adjusted pressure threshold 324 may be dynamically set based on the detection of other conditions that may contribute to an occlusion. For example, as will be described further, the adjusted pressure threshold 324 may be set and/or adjusted based on air detected in a fluid infusion line (e.g., the fluid infusion line 21 of FIG. 1). As another example, the algorithm of the subject technology may, on detecting a pressure decay within the fluid infusion line, begin to determine whether air is also being introduced into the line using a sensor (e.g., the sensor 84 of FIG. 2). On detecting a predetermined amount of air or amount of diffusion of air (see, e.g., threshold 314 of FIG. 3B), the algorithm may switch to using threshold 324 to trigger an alarm.

Time period 325 represents the amount of time between the occurrence of the example upstream occlusion and the triggering of a pressure alarm under default conditions (e.g., using threshold 323). According to some implementations, the TTA (time-to-alarm) will be shortened dynamically based on certain conditions, such as, as shown in the depicted graph, when threshold 324 is used.

FIG. 3B depicts a graph with example curves representative of an amount of air present in a fluid infusion line (e.g., fluid infusion line 21 of FIG. 1) under normal and occluded conditions, including thresholds for detecting the same, according to various aspects of the subject technology. Silicon tubing is porous to air under vacuum or semi-vacuum conditions. When an upstream occlusion occurs, and the pump is pumping against a collapsed tubing, a vacuum may be created within the tubing, causing air to diffuse into the tubing and/or fluid through the silicon walls, and thereby forming air bubbles in the tubing and/or fluid. As described previously, a processor (e.g., within the infusion device 10 of FIGS. 1 and 2) may detect the amount of air within an infusion line using a sensor (e.g., the sensor 84 of FIG. 2).

The x-axis of the depicted graph represents time, and the y-axis represents an amount of air in the fluid infusion line. Curve 311 represents a detected amount of air within an infusion line under normal conditions (e.g., in the absence of an upstream occlusion). As depicted, the detected amount of air has minimal variation in the absence of an occlusion. Curve 312, on the other hand, represents an example variation in the amount of air within an infusion line during an upstream occlusion. In the depicted example, an upstream occlusion has occurred and the amount of air increases over time, represented by the increasing slope depicted in curve 312.

A default air-in-line threshold 313 represents a threshold for triggering an air-in-line alarm under normal conditions. For example, default air-in-line threshold 313 may be preprogrammed into the infusion device 10 by the time infusion is initiated. When the amount of air reaches the default air-in-line threshold 313, an alarm is triggered to notify clinicians that there is air in the fluid infusion line 21. While the infusion device 10 may include both an occlusion alarm and an air-in-line alarm, in some implementations the air-in-line alarm may be interpreted as an occlusion alarm.

Secondary air-in-line threshold 314, on the other hand, represents a secondary threshold that may be used instead of the default air-in-line threshold 313 under certain conditions. For example, similar to the pressure examples in FIG. 3A, the secondary threshold 314 may be used when a pressure sensed by a pressure sensor (e.g., the upstream pressure sensor 80 of FIG. 2) detects a predetermined pressure decay over time and/or reaches a certain pressure (e.g., the adjusted pressure threshold of FIG. 3A). While the secondary air-in-line threshold 314 is depicted in FIG. 3A as being less than threshold 313, in some implementations, the secondary threshold 314 is greater than or equal to the threshold 313.

Time period 315 represents the amount of time between the occurrence of the example upstream occlusion and the triggering of an air-in-line alarm under default conditions (e.g., using threshold 313). According to some implementations, this TTA (time-to-alarm) will be shortened dynamically based on certain conditions, such as, as shown in the depicted graph, when threshold 314 is used.

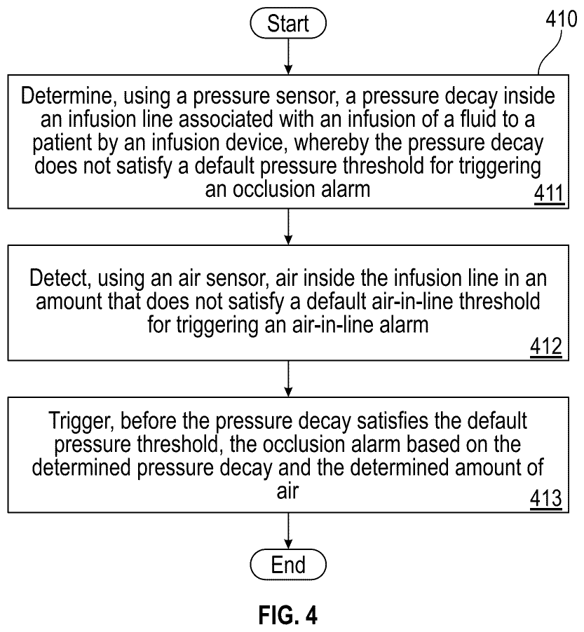

FIG. 4 depicts a first example process 410 for triggering an occlusion alarm, according to aspects of the subject technology. One or more blocks of process 410 may be implemented, for example, by one or more computing devices including, for example, the infusion device 10. In some implementations, one or more of the blocks may be implemented based on one or more machine learning algorithms. In some implementations, one more of the blocks may be implemented apart from other blocks, and by one or more different processors or devices. Further, for explanatory purposes, the blocks of example process 410 are described as occurring in serial, or linearly. However, multiple blocks of example process 410 may occur in parallel. Additionally, the blocks of example process 410 need not be performed in the order shown and one or more of the blocks of example process 410 need not be performed.

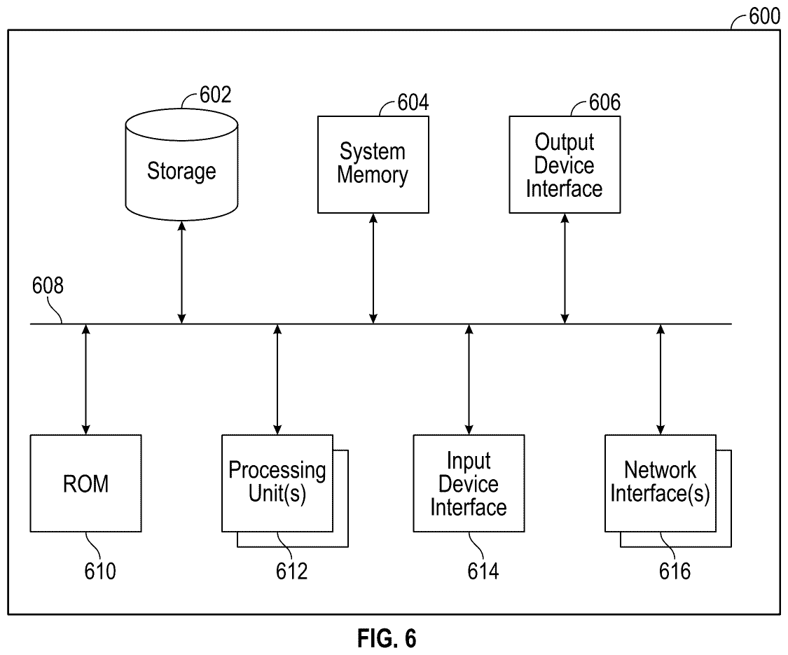

An algorithm executed by a processor associated with the infusion device 10 (see, e.g., FIG. 6) continuously monitors pressure values using a pressure sensor (e.g., the upstream pressure sensor 80, or the downstream pressure sensor 82 of FIG. 2) to determine whether the current pressure within the liquid infusion line 21 satisfies a default predetermined (high or low) pressure limit for triggering an occlusion alarm (e.g., default pressure threshold of FIG. 3A). In process 410, the algorithm determines a pressure decay inside a fluid infusion line 21 associated with an infusion of a fluid to a patient by an infusion device 10, whereby the pressure decay does not satisfy a default pressure threshold for triggering an occlusion alarm (411). In some implementations, the determined pressure decay may include a pressure value measured by a pressure sensor (e.g., the upstream pressure sensor 80 of FIG. 2), and the default pressure threshold may be a minimum pressure value for triggering the occlusion alarm. With brief reference to FIG. 3A, the currently detected pressure may be less than the adjusted pressure threshold 324 but, in this example, has not reached default pressure threshold 323. Thus, the occlusion alarm may not be triggered based on the pressure reading alone. In some implementations, the determined pressure decay may be a slope value of a decay in pressure over time. In such implementations, the default pressure value may include fixed pressure value or may be a maximum slope value for the decay in pressure over time. In some implementations, pressure decay be a rate of change, and the thresholds described herein may be the rate of change in the pressure over a period of time.

In the depicted example, the algorithm detects, using an air sensor 84, air inside the infusion line in an amount that does not satisfy a default air-in-line threshold for triggering an air-in-line alarm (412). For example, the processor of the infusion device 10 may continuously monitor sensor values provided by air-in-line sensor 84 for a predetermined threshold amount of air. With brief reference to FIG. 3B, the amount of air may be more than the secondary air-in-line threshold 314 but, in this example, has not reached the default air-in-line threshold 313. In some implementations, the amount of air is measured as the volume of air in the line. In some implementations, the amount of air is measured as the number of air bubbles in the line.

Both of the foregoing pressure and air conditions are indicative of a potential occlusion within the infusion line; however, neither condition alone may be sufficient to trigger an alarm. The algorithm of the subject technology observes the pressure decay in addition to increasing air within the infusion line and, before the pressure has decayed enough to satisfy the default pressure threshold, the algorithm triggers the occlusion alarm based on the determined amount of air and the determined pressure decay (413). In this regard, the algorithm utilizes thresholds that are below thresholds normally utilized to trigger any particular alarm.

The thresholds and measurements (e.g., by pressure or air sensors) described herein may relate to fixed values at a single point in time, or they may be an average or mean value for a predetermined period of time. In some implementations, these thresholds and measurements may be a respective value indicative of a slope or rate of change for the corresponding parameter (air or pressure). In one example, the algorithm may utilize adjusted pressure threshold 324, which may be a predetermined percentage (e.g., 80%, 70%, 60%, 50%, etc.) of the default pressure threshold 323 (e.g., a default threshold programmed into the infusion device 10). In some implementations, a threshold for one parameter is adjusted proportionally based on the value of the other parameter with respect to the threshold for the other parameter. For example, as a detected amount of air gets closer to the default air-in-line threshold, the adjusted pressure threshold may change (e.g., become greater) towards a value that is more easily triggered.

Similarly, the algorithm may utilize the described secondary air-in-line threshold 314, which may be a predetermined percentage of the default air-in-line threshold 313 (e.g., a default threshold programmed into the infusion device 10). In some implementations, thresholds 314 and 324 (and/or thresholds 313, 323) may include predetermined slope values (or, in some implementations, a rate of change). For example, if thresholds 313 and 323 have not been met, but a slope greater than a predetermined slope for one of the parameters is detected for more than a predetermined amount of time before the threshold is met then the alarm may be triggered. In some implementations, thresholds 314, 324 (and/or thresholds 313, 323) may include predetermined rates of change. By reducing the alarm limits of each factor (e.g., pressure and air), the time to occlusion alarm may be faster than when relying on satisfying one or both default alarms.

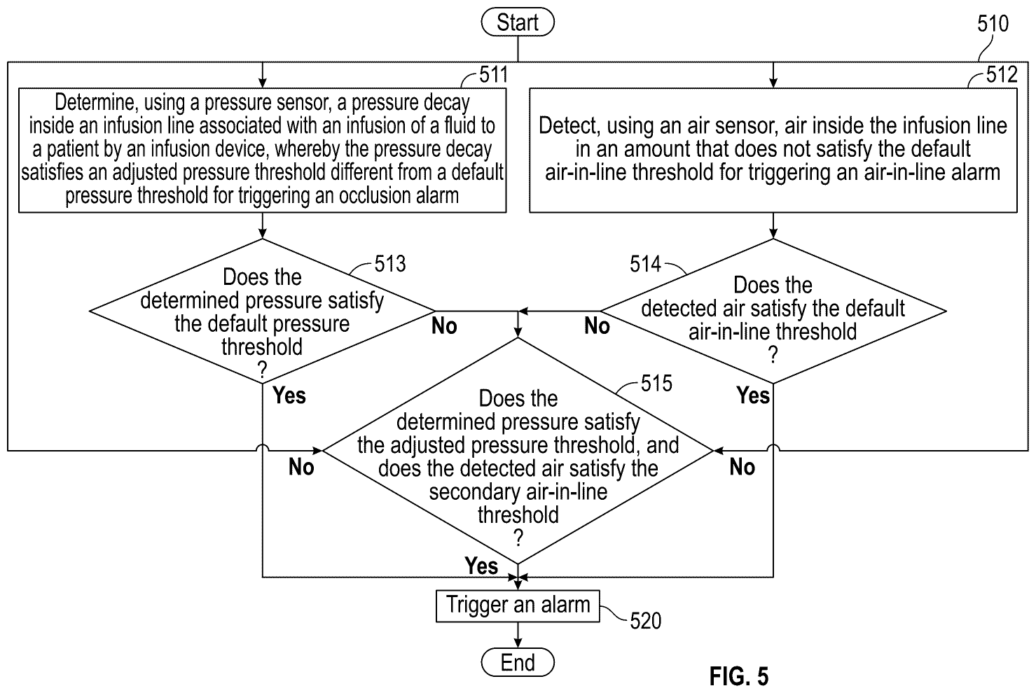

FIG. 5 depicts a second example process 510 for triggering an occlusion alarm, according to aspects of the subject technology. According to various implementations, process 510 operates similar to and/or may replace or augment process 410. One or more blocks of process 510 may be implemented, for example, by one or more computing devices including, for example, infusion device 10. In some implementations, one or more of the blocks may be implemented based on one or more machine learning algorithms. In some implementations, one more of the blocks may be implemented apart from other blocks, and by one or more different processors or devices. Further, for explanatory purposes, the blocks of example process 510 are described as occurring in serial, or linearly. However, multiple blocks of example process 510 may occur in parallel. Additionally, the blocks of example process 510 need not be performed in the order shown and one or more of the blocks of example process 510 need not be performed.

In example process 510, the algorithm determines, using pressure values obtained from pressure sensor 84, a pressure decay inside an infusion line associated with an infusion of a fluid to a patient by an infusion device, whereby the pressure decay satisfies an adjusted pressure threshold different from a default pressure threshold for triggering an occlusion alarm (511). With reference to FIGS. 3A and 5, if the pressure decay detected by the pressure sensor 80, 82 satisfies threshold 323 (e.g., the pressure value resulting from a decay in pressure) then the occlusion alarm may be triggered. If the detected pressure decay does not satisfy the thresholds 323 and 324 then the process returns to monitoring air and pressure (using pressure sensor(s) 80, 82 and air sensor 84). (Start.) If the detected pressure satisfies threshold 324, but not the threshold 323, the algorithm determines the status of air within the infusion line to determine whether an occlusion alarm should be triggered. The algorithm triggers an occlusion alarm responsive to both threshold 324 and threshold 314 being satisfied.

The algorithm detects, using an air sensor 84, air inside the infusion line in an amount that does not satisfy the default air-in-line threshold for triggering an air-in-line alarm (512). With reference to FIGS. 3B and 5, if an amount of air detected by air sensor 84 satisfies threshold 313 then an air-in-line alarm may be triggered. If the detected amount of air does not satisfy thresholds 313 and 314 then the process returns to monitoring air and pressure (using pressure sensor(s) 80, 82 and air sensor 84). (Start.) In some implementations, if the detected amount of air satisfies threshold 314, but not the threshold 313, then the algorithm triggers an occlusion alarm on determining that a pressure decay exists and meets certain criteria, such as satisfying threshold 324. The algorithm triggers an occlusion alarm responsive to both threshold 324 and threshold 314 being satisfied (515, 520).

While the disclosure relates to an occlusion alarm, generally, based on detected pressure and air, in some implementations, the air-in-line alarm may be triggered early based on the foregoing parameters and thresholds. For example, if the detected amount of air does not satisfy thresholds 313 and 314 then the process returns to monitoring air and pressure (using pressure sensor(s) 80, 82 and air sensor 84). (Start.) However, if the detected amount of air satisfies threshold 314, but not the threshold 313, then the algorithm may trigger the air-in-line alarm on determining that a pressure decay exists and meets certain criteria, such as satisfying threshold 324 (515, 520). In some implementations, the infusion device 10 may be configured to trigger both an air-in-line alarm (based on air) and an occlusion alarm (based on pressure). The algorithm may be configured to switch from throwing an air-in-line alarm to the upstream occlusion alarm based on the detected amount of air satisfying threshold 314, but not the threshold 313, and the pressure decay satisfying threshold 324.

According to some implementations, before the occlusion alarm is triggered, the secondary air-in-line threshold is adjusted dynamically based on the determined pressure within the infusion line. For example, in some implementations, the secondary air-in-line threshold is adjusted when the determined pressure is at or below a predetermined amount. As another example, in some implementations, the secondary air-in-line threshold may be scaled up or down based on the determined pressure.

In some implementations, before the occlusion alarm is triggered, the secondary air-in-line threshold is adjusted based on a flow rate of the fluid inside the infusion line. For example, the secondary air-in-line threshold may be adjusted (e.g., to trigger an occlusion alarm faster) when the flow rate of the fluid inside the infusion line is less than 1 milliliter per hour. As another example, the secondary air-in-line threshold may be adjusted (e.g., to trigger an occlusion alarm faster) when the flow rate of the fluid inside the infusion line is less than 0.1 milliliters per hour. In some implementations, the secondary air-in-line threshold is dynamically set based on a multiplication of the threshold by a factor that is determined based on the flow rate of the fluid inside the fluid infusion line 21. For example, in some implementations, when the flow rate is 1 milliliter per hour, the factor is one-half. Accordingly, the secondary air-in-line threshold is set to one-half of the default air-in-line threshold (e.g., the default air-in-line threshold, multiplied by a factor of one-half) when the flow rate is less than or equal to 1 milliliter per hour. As another example, in some implementations, the factor is one-quarter when the flow rate is 0.1 milliliters per hour. At a flow rate of 0.1 milliliters per hour, the secondary air-in-line threshold would then be set to one-quarter of the default air-in-line threshold.

In some implementations, before the occlusion alarm is triggered, the adjusted pressure threshold is dynamically set based on the determined amount of air. For example, the adjusted pressure threshold may be adjusted when the determined amount of air is at or above a predetermined amount. As another example, the adjusted pressure threshold may be scaled up or down based on the determined amount of air.

In some implementations, before the occlusion alarm is triggered, the adjusted pressure threshold is dynamically set based on the flow rate of the fluid inside the infusion line. For example, the adjusted pressure threshold may be dynamically set when the flow rate of the fluid inside the infusion line is less than 1 milliliter per hour. As another example, the adjusted pressure threshold may be dynamically set when the flow rate of the fluid inside the infusion line is less than 0.1 milliliter per hour. In some implementations, the adjusted pressure threshold is dynamically set based on a multiplication of the threshold by a factor that is determined based on the flow rate of the fluid inside the fluid infusion line 21. For example, in some implementations, when the flow rate is 1 milliliter per hour, the factor is two. Accordingly, the adjusted pressure threshold is set to two times the default pressure threshold (e.g., the default air-in-line threshold, multiplied by a factor of two) when the flow rate is less than or equal to 1 milliliter per hour. As another example, in some implementations, the factor is four when the flow rate is 0.1 milliliters per hour. At a flow rate of 0.1 milliliters per hour, the adjusted pressure threshold would then be set to four times the default pressure threshold. As another example, the adjusted pressure threshold may be scaled up or down based on the determined amount of air.

According to various implementations, the disclosed algorithm may be configured to disable the infusion device 10, or otherwise terminate an ongoing infusion or administration of fluid to a patient responsive to the conditions described herein for trigging the occlusion alarm or, in some implementations, the air-in-line alarm. In this regard, the steps of triggering the alarm 413 and 520 of respective processes 410 and 510 may be replaced or supplemented with disabling the pump or terminating the infusion. In some implementations, disabling the pump and/or terminating the infusion may further include locking the user interface associated with a pump so that a clinician cannot reactivate the pump or infusion without acknowledging the corresponding alarm and/or providing authorization based on elevated access rights (e.g., that of a supervisor).

Many of the above-described example processes 410, 510, and related features and applications, may also be implemented as software processes that are specified as a set of instructions recorded on a computer readable storage medium (also referred to as computer readable medium), and may be executed automatically (e.g., without user intervention). When these instructions are executed by one or more processing unit(s) (e.g., one or more processors, cores of processors, or other processing units), they cause the processing unit(s) to perform the actions indicated in the instructions. Examples of computer readable media include, but are not limited to, CD-ROMs, flash drives, RAM chips, hard drives, EPROMs, etc. The computer readable media does not include carrier waves and electronic signals passing wirelessly or over wired connections.

The term “software” is meant to include, where appropriate, firmware residing in read-only memory or applications stored in magnetic storage, which can be read into memory for processing by a processor. Also, in some implementations, multiple software aspects of the subject disclosure can be implemented as sub-parts of a larger program while remaining distinct software aspects of the subject disclosure. In some implementations, multiple software aspects can also be implemented as separate programs. Finally, any combination of separate programs that together implement a software aspect described herein is within the scope of the subject disclosure. In some implementations, the software programs, when installed to operate on one or more electronic systems, define one or more specific machine implementations that execute and perform the operations of the software programs.

A computer program (also known as a program, software, software application, script, or code) can be written in any form of programming language, including compiled or interpreted languages, declarative or procedural languages, and it can be deployed in any form, including as a stand-alone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

FIG. 6 is a conceptual diagram illustrating an example electronic system 600 for detecting an upstream occlusion, according to aspects of the subject technology. Electronic system 600 may be implemented by a computing device for execution of software associated with one or more portions or steps of processes 410 and/or 510, or components and methods provided by FIGS. 1-5. In this regard, electronic system 600 may include the infusion device 10, and/or a computing device within or connected to the infusion device 10.

Electronic system 600 may include various types of computer readable media and interfaces for various other types of computer readable media. In the depicted example, electronic system 600 includes a bus 608, processing unit(s) 612, a system memory 604, a read-only memory (ROM) 610, a permanent storage device 602, an input device interface 614, an output device interface 606, and one or more network interfaces 616. In some implementations, electronic system 600 may include or be integrated with other computing devices or circuitry for operation of the various components and methods previously described.

Bus 608 collectively represents all system, peripheral, and chipset buses that communicatively connect the numerous internal devices of electronic system 600. For instance, bus 408 communicatively connects processing unit(s) 612 with ROM 610, system memory 604, and permanent storage device 602.

From these various memory units, processing unit(s) 612 retrieves instructions to execute and data to process, in order to execute the processes of the subject disclosure. The processing unit(s) can be a single processor or a multi-core processor in different implementations.

ROM 610 stores static data and instructions that are needed by processing unit(s) 612 and other modules of the electronic system. Permanent storage device 602, on the other hand, is a read-and-write memory device. This device is a non-volatile memory unit that stores instructions and data even when electronic system 600 is off. Some implementations of the subject disclosure use a mass-storage device (such as a magnetic or optical disk and its corresponding disk drive) as permanent storage device 602.

Other implementations use a removable storage device (such as a floppy disk, flash drive, and its corresponding disk drive) as permanent storage device 602. Like permanent storage device 602, system memory 604 is a read-and-write memory device. However, unlike storage device 602, system memory 604 is a volatile read-and-write memory, such as random-access memory. System memory 604 stores some of the instructions and data that the processor needs at runtime. In some implementations, the processes of the subject disclosure are stored in system memory 604, permanent storage device 602, and/or ROM 610. From these various memory units, processing unit(s) 612 retrieves instructions to execute and data to process, in order to execute the processes of some implementations.

Bus 608 also connects to input and output device interfaces 614 and 606. Input device interface 614 enables the user to communicate information and select commands to the electronic system. Input devices used with input device interface 614 include, e.g., alphanumeric keyboards and pointing devices (also called “cursor control devices”). Output device interfaces 606 enables, e.g., the display of images generated by the electronic system 600. Output devices used with output device interface 606 include, e.g., printers and display devices, such as cathode ray tubes (CRT) or liquid crystal displays (LCD). Some implementations include devices such as a touchscreen that functions as both input and output devices.

Also, as shown in FIG. 6, bus 608 also couples electronic system 600 to a network (not shown) through network interfaces 616. Network interfaces 616 may include, e.g., a wireless access point (e.g., Bluetooth or Wi-Fi) or radio circuitry for connecting to a wireless access point. Network interfaces 616 may also include hardware (e.g., Ethernet hardware) for connecting the computer to a part of a network of computers such as a local area network (“LAN”), a wide area network (“WAN”), wireless LAN, or an Intranet, or a network of networks, such as the Internet. Any or all components of electronic system 600 can be used in conjunction with the subject disclosure.

These functions described above can be implemented in computer software, firmware, or hardware. The techniques can be implemented using one or more computer program products. Programmable processors and computers can be included in or packaged as mobile devices. The processes and logic flows can be performed by one or more programmable processors and by one or more programmable logic circuitry. General and special purpose computing devices and storage devices can be interconnected through communication networks.

Some implementations include electronic components, such as microprocessors, storage and memory that store computer program instructions in a machine-readable or computer-readable medium (also referred to as computer-readable storage media, machine-readable media, or machine-readable storage media). Some examples of such computer-readable media include RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, read-only and recordable Blu-Ray® discs, ultra density optical discs, any other optical or magnetic media, and floppy disks. The computer-readable media can store a computer program that is executable by at least one processing unit and includes sets of instructions for performing various operations. Examples of computer programs or computer code include machine code, such as is produced by a compiler, and files including higher-level code that are executed by a computer, an electronic component, or a microprocessor using an interpreter.

While the above discussion primarily refers to microprocessor or multi-core processors that execute software, some implementations are performed by one or more integrated circuits, such as application specific integrated circuits (ASICs) or field programmable gate arrays (FPGAs). In some implementations, such integrated circuits execute instructions that are stored on the circuit itself.

As used in this specification and any claims of this application, the terms “computer”, “server”, “processor”, and “memory” all refer to electronic or other technological devices. These terms exclude people or groups of people. For the purposes of the specification, the terms display or displaying means displaying on an electronic device. As used in this specification and any claims of this application, the terms “computer readable medium” and “computer readable media” are entirely restricted to tangible, physical objects that store information in a form that is readable by a computer. These terms exclude any wireless signals, wired download signals, and any other ephemeral signals.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; e.g., feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user, e.g., by sending web pages to a web browser on a user's client device in response to requests received from the web browser.

Implementations of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other and may interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

Those of skill in the art would appreciate that the various illustrative blocks, modules, elements, components, methods, and algorithms described herein may be implemented as electronic hardware, computer software, or combinations of both. To illustrate this interchangeability of hardware and software, various illustrative blocks, modules, elements, components, methods, and algorithms have been described above generally in terms of their functionality. Whether such functionality is implemented as hardware or software, depends upon the particular application and design constraints imposed on the overall system. The described functionality may be implemented in varying ways for each particular application. Various components and blocks may be arranged differently (e.g., arranged in a different order, or partitioned in a different way) all without departing from the scope of the subject technology.

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order and are not meant to be limited to the specific order or hierarchy presented.

### Illustration of Subject Technology as Clauses:

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identifications.

Clause 1. A method, comprising: determining, using a pressure sensor, a pressure decay inside an infusion line associated with an infusion of a fluid to a patient by an infusion device, whereby the pressure decay does not satisfy a default pressure threshold for triggering an occlusion alarm; detecting, using an air sensor, air inside the infusion line in an amount that does not satisfy a default air-in-line threshold for triggering an air-in-line alarm; and triggering, before the pressure decay satisfies the default pressure threshold, the occlusion alarm based on the determined pressure decay and the detected amount of air.

Clause 2. The method of claim 1, wherein the detected amount of air comprises one of a volume of air in the line or a number of air bubbles in the line.

Clause 3. The method of claim 1, wherein the pressure decay is determined as occurring upstream from the infusion device and the occlusion alarm comprises an upstream occlusion alarm.

Clause 4. The method of claim 1, wherein triggering the occlusion alarm comprises: setting an adjusted pressure threshold for triggering the infusion alarm based on the detected amount of air satisfying a secondary air-in-line threshold different from the default air-in-line threshold, wherein the adjusted pressure threshold is different from the default pressure threshold; and triggering the occlusion alarm based on the adjusted pressure threshold.

Clause 5. The method of claim 4, wherein the adjusted pressure threshold is lower than the default pressure threshold and the amount of air satisfying the secondary air-in-line threshold comprises the detected amount of air becoming greater than the secondary air-in-line threshold but lower than the default air-in-line threshold.

Clause 6. The method of claim 4, wherein the pressure decay comprises a current pressure value and the default pressure threshold is a minimum pressure value for triggering the occlusion alarm.

Clause 7. The method of claim 4, wherein the secondary air-in-line threshold comprises a rate of change in the amount of air detected over a period of time; or the adjusted pressure threshold comprises a rate of change in the pressure decay over the period of time.

Clause 8. The method of claim 4, further comprising, before triggering the occlusion alarm: adjusting the secondary air-in-line threshold based on the determined pressure decay; or adjusting the adjusted pressure threshold based on the detected amount of air.

Clause 9. The method of claim 4, further comprising, before triggering the occlusion alarm: adjusting, based on a flow rate of the fluid inside the infusion line, at least one of the adjusted pressure threshold and the secondary air-in-line threshold.

Clause 10. The method of claim 4, wherein the adjusted pressure threshold is set to a predetermined percentage of the default pressure threshold or the secondary air-in-line threshold is set to a predetermined percentage of the default air-in-line threshold.

Clause 11. An infusion pump, comprising: one or more occluder valves comprising a lower occluder valve, the lower occluder valve configured to, when activated, cause a compression of a fluid tubing loaded into the infusion pump, the compression fluidically isolating a downstream portion of the fluid tubing from an upstream portion of the fluid tubing when the lower occluder valve is operating under a normal operating condition; an air-in-line sensor; a pressure sensor; an occlusion alarm; and a processor configured to: determine, using a pressure sensor, a pressure decay inside an infusion line associated with an infusion of a fluid to a patient by an infusion device, whereby the pressure decay does not satisfy a default pressure threshold for triggering the occlusion alarm; detect, using the air-in-line sensor, air inside the infusion line in an amount that does not satisfy a default air-in-line threshold for triggering an air-in-line alarm; and trigger, before the pressure decay satisfies the default pressure threshold, the occlusion alarm based on the determined pressure decay and the detected amount of air.

Clause 12. The infusion pump of claim 11, wherein triggering the occlusion alarm comprises: setting an adjusted pressure threshold for triggering the infusion alarm based on the detected amount of air satisfying a secondary air-in-line threshold different from the default air-in-line threshold, wherein the adjusted pressure threshold is different from the default pressure threshold; and triggering the occlusion alarm based on the adjusted pressure threshold.

Clause 13. The infusion pump of claim 12, wherein the processor is further configured to: before triggering the occlusion alarm: adjust the secondary air-in-line threshold based on the determined pressure; and adjust the adjusted pressure threshold based on the detected amount of air.

Clause 14. The infusion pump of claim 12, wherein the processor is further configured to: before triggering the occlusion alarm: adjust, based on a flow rate of the fluid inside the infusion line, at least one of the adjusted pressure threshold and secondary air-in-line threshold.

Clause 15. The infusion pump of claim 12, wherein the adjusted pressure threshold is set to a predetermined percentage of the default pressure threshold or the secondary air-in-line threshold is set to a predetermined percentage of the default air-in-line threshold.

Clause 16. A non-transitory, computer-readable medium comprising instructions that, when executed by one or more processors, cause the one or more processors to: determine, using a pressure sensor, a pressure decay inside an infusion line associated with an infusion of a fluid to a patient by an infusion device, whereby the pressure decay does not satisfy a default pressure threshold for triggering an occlusion alarm; detect, using an air sensor, air inside the infusion line in an amount that does not satisfy a default air-in-line threshold for triggering an air-in-line alarm; and trigger, before the pressure decay satisfies the default pressure threshold, the occlusion alarm based on the determined pressure decay and the detected amount of air.

Clause 17. The non-transitory, computer-readable medium of claim 16, wherein triggering the occlusion alarm comprises: setting an adjusted pressure threshold for triggering the infusion alarm based on the detected amount of air satisfying a secondary air-in-line threshold different from the default air-in-line threshold, wherein the adjusted pressure threshold is different from the default pressure threshold; and triggering the occlusion alarm based on the adjusted pressure threshold.

Clause 18. The non-transitory, computer-readable medium of claim 17, further comprising instructions that, when executed, cause the one or more processors to: before triggering the occlusion alarm: adjust the secondary air-in-line threshold based on the determined pressure decay; and adjust the adjusted pressure threshold based on the detected amount of air.

Clause 19. The non-transitory, computer-readable medium of claim 17, further comprising instructions that, when executed, cause the one or more processors to: before triggering the occlusion alarm: adjust, based on a flow rate of the fluid inside the infusion line, at least one of the adjusted pressure threshold and the secondary air-in-line threshold.

Clause 20. The non-transitory, computer-readable medium of claim 17, wherein the adjusted pressure is set to a predetermined percentage of the default pressure threshold or the secondary air-in-line threshold is set to a predetermined percentage of the default air-in-line threshold.

### Further Consideration

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

The previous description is provided to enable any person skilled in the art to practice the various aspects described herein. The previous description provides various examples of the subject technology, and the subject technology is not limited to these examples. Various modifications to these aspects will be readily apparent to those skilled in the art, and the generic principles defined herein may be applied to other aspects. Thus, the claims are not intended to be limited to the aspects shown herein but are to be accorded the full scope consistent with the language of the claims, wherein reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention described herein.

The predicate words “configured to,” “operable to,” and “programmed to” do not imply any particular tangible or intangible modification of a subject, but, rather, are intended to be used interchangeably. For example, a processor configured to monitor and control an operation or a component, may also mean the processor being programmed to monitor and control the operation or the processor being operable to monitor and control the operation. Likewise, a processor configured to execute code can be construed as a processor programmed to execute code or operable to execute code.

The term “automatic,” as used herein, may include performance by a computer or machine without user intervention, for example, by instructions responsive to a predicate action by the computer or machine or other initiation mechanism. The word “example” is used herein to mean “serving as an example or illustration.” Any aspect or design described herein as “example” is not necessarily to be construed as preferred or advantageous over other aspects or designs.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “implementation” does not imply that such implementation is essential to the subject technology or that such implementation applies to all configurations of the subject technology. A disclosure relating to an implementation may apply to all implementations, or one or more implementations. An implementation may provide one or more examples. A phrase such as an “implementation” may refer to one or more implementations and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such as a “configuration” may refer to one or more configurations and vice versa.

As used herein a “user interface” (also referred to as an interactive user interface, a graphical user interface or a UI) may refer to a network-based interface including data fields and/or other control elements for receiving input signals or providing electronic information and/or for providing information to the user in response to any received input signals. Control elements may include dials, buttons, icons, selectable areas, or other perceivable indicia presented via the UI that, when interacted with (e.g., clicked, touched, selected, etc.), initiates an exchange of data for the device presenting the UI. A UI may be implemented in whole or in part using technologies such as hyper-text mark-up language (HTML), FLASH™, JAVA™, .NET™, C, C++, web services, or rich site summary (RSS). In some implementations, a UI may be included in a stand-alone client (for example, thick client, fat client) configured to communicate (e.g., send or receive data) in accordance with one or more of the aspects described. The communication may be to or from a medical device or server in communication therewith.

As used herein, the terms “determine” or “determining” encompass a wide variety of actions. For example, “determining” may include calculating, computing, processing, deriving, generating, obtaining, looking up (e.g., looking up in a table, a database, or another data structure), ascertaining and the like via a hardware element without user intervention. Also, “determining” may include receiving (e.g., receiving information), accessing (e.g., accessing data in a memory) and the like via a hardware element without user intervention. “Determining” may include resolving, selecting, choosing, establishing, and the like via a hardware element without user intervention.

As used herein, the terms “provide” or “providing” encompass a wide variety of actions. For example, “providing” may include storing a value in a location of a storage device for subsequent retrieval, transmitting a value directly to the recipient via at least one wired or wireless communication medium, transmitting or storing a reference to a value, and the like. “Providing” may also include encoding, decoding, encrypting, decrypting, validating, verifying, and the like via a hardware element.

As used herein, the term “message” encompasses a wide variety of formats for communicating (e.g., transmitting or receiving) information. A message may include a machine-readable aggregation of information such as an XML document, fixed field message, comma separated message, JSON, a custom protocol, or the like. A message may, in some implementations, include a signal utilized to transmit one or more representations of the information. While recited in the singular, it will be understood that a message may be composed, transmitted, stored, received, etc. in multiple parts.

As used herein, the term “selectively” or “selective” may encompass a wide variety of actions. For example, a “selective” process may include determining one option from multiple options. A “selective” process may include one or more of: dynamically determined inputs, preconfigured inputs, or user-initiated inputs for making the determination. In some implementations, an n-input switch may be included to provide selective functionality where n is the number of inputs used to make the selection.

As user herein, the terms “satisfy” or “correspond” or “corresponding” encompass a structural, functional, quantitative and/or qualitative correlation or relationship between two or more objects, data sets, information and/or the like, preferably where the correspondence or relationship may be used to translate one or more of the two or more objects, data sets, information and/or the like so to appear to be the same or equal. Correspondence may be assessed using one or more of a threshold, a value range, fuzzy logic, pattern matching, a machine learning assessment model, or combinations thereof.

In some implementations, data generated or detected can be forwarded to a “remote” device or location, where “remote,” means a location or device other than the location or device at which the program is executed. For example, a remote location could be another location (e.g., office, lab, etc.) in the same city, another location in a different city, another location in a different state, another location in a different country, etc. As such, when one item is indicated as being “remote” from another, what is meant is that the two items can be in the same room but separated, or at least in different rooms or different buildings, and can be at least one mile, ten miles, or at least one hundred miles apart. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. Examples of communicating media include radio or infra-red transmission channels as well as a network connection to another computer or networked device, and the internet or including email transmissions and information recorded on websites and the like.

What is claimed is:

1. A method, comprising:
determining, using a pressure sensor, a pressure decay inside an infusion line associated with an infusion of a fluid to a patient by an infusion device, whereby the pressure decay does not satisfy a default pressure threshold for triggering an occlusion alarm;
detecting, using an air sensor, air inside the infusion line in an amount that does not satisfy a default air-in-line threshold for triggering an air-in-line alarm; and
triggering, before the pressure decay satisfies the default pressure threshold, the occlusion alarm based on the determined pressure decay and the detected amount of air.

2. The method of claim 1, wherein the detected amount of air comprises one of a volume of air in the line or a number of air bubbles in the line.

3. The method of claim 1, wherein the pressure decay is determined as occurring upstream from the infusion device and the occlusion alarm comprises an upstream occlusion alarm.

4. The method of claim 1, wherein triggering the occlusion alarm comprises:
setting an adjusted pressure threshold for triggering the infusion alarm based on the detected amount of air satisfying a secondary air-in-line threshold different from the default air-in-line threshold, wherein the adjusted pressure threshold is different from the default pressure threshold; and
triggering the occlusion alarm based on the adjusted pressure threshold.

5. The method of claim 4, wherein the adjusted pressure threshold is lower than the default pressure threshold and the amount of air satisfying the secondary air-in-line threshold comprises the detected amount of air becoming greater than the secondary air-in-line threshold but lower than the default air-in-line threshold.

6. The method of claim 4, wherein the pressure decay comprises a current pressure value and the default pressure threshold is a minimum pressure value for triggering the occlusion alarm.

7. The method of claim 4, wherein
the secondary air-in-line threshold comprises a rate of change in the amount of air detected over a period of time; or
the adjusted pressure threshold comprises a rate of change in the pressure decay over the period of time.

8. The method of claim 4, further comprising, before triggering the occlusion alarm:
adjusting the secondary air-in-line threshold based on the determined pressure decay; or
adjusting the adjusted pressure threshold based on the detected amount of air.

9. The method of claim 4, further comprising, before triggering the occlusion alarm:
adjusting, based on a flow rate of the fluid inside the infusion line, at least one of the adjusted pressure threshold and the secondary air-in-line threshold.

10. The method of claim 4, wherein the adjusted pressure threshold is set to a predetermined percentage of the default pressure threshold or the secondary air-in-line threshold is set to a predetermined percentage of the default air-in-line threshold.

11. An infusion pump, comprising:
one or more occluder valves comprising a lower occluder valve, the lower occluder valve configured to, when activated, cause a compression of a fluid tubing loaded into the infusion pump, the compression fluidically isolating a downstream portion of the fluid tubing from an upstream portion of the fluid tubing when the lower occluder valve is operating under a normal operating condition;
an air-in-line sensor;
a pressure sensor; and
a processor configured to:
determine, using a pressure sensor, a pressure decay inside an infusion line associated with an infusion of a fluid to a patient by an infusion device, whereby the pressure decay does not satisfy a default pressure threshold for triggering an occlusion alarm;
detect, using the air-in-line sensor, air inside the infusion line in an amount that does not satisfy a default air-in-line threshold for triggering an air-in-line alarm; and
trigger, before the pressure decay satisfies the default pressure threshold, the occlusion alarm based on the determined pressure decay and the detected amount of air.

12. The infusion pump of claim 11, wherein triggering the occlusion alarm comprises:
setting an adjusted pressure threshold for triggering the infusion alarm based on the detected amount of air satisfying a secondary air-in-line threshold different from the default air-in-line threshold, wherein the adjusted pressure threshold is different from the default pressure threshold; and
triggering the occlusion alarm based on the adjusted pressure threshold.

13. The infusion pump of claim 12, wherein the processor is further configured to:
before triggering the occlusion alarm:
adjust the secondary air-in-line threshold based on the determined pressure; and
adjust the adjusted pressure threshold based on the detected amount of air.

14. The infusion pump of claim 12, wherein the processor is further configured to:
before triggering the occlusion alarm:
adjust, based on a flow rate of the fluid inside the infusion line, at least one of the adjusted pressure threshold and secondary air-in-line threshold.

15. The infusion pump of claim 12, wherein the adjusted pressure threshold is set to a predetermined percentage of the default pressure threshold or the secondary air-in-line threshold is set to a predetermined percentage of the default air-in-line threshold.

16. A non-transitory, computer-readable medium comprising instructions that, when executed by one or more processors, cause the one or more processors to:
determine, using a pressure sensor, a pressure decay inside an infusion line associated with an infusion of a fluid to a patient by an infusion device, whereby the pressure decay does not satisfy a default pressure threshold for triggering an occlusion alarm;
detect, using an air sensor, air inside the infusion line in an amount that does not satisfy a default air-in-line threshold for triggering an air-in-line alarm; and
trigger, before the pressure decay satisfies the default pressure threshold, the occlusion alarm based on the determined pressure decay and the detected amount of air.

17. The non-transitory, computer-readable medium of claim 16, wherein triggering the occlusion alarm comprises:
setting an adjusted pressure threshold for triggering the infusion alarm based on the detected amount of air satisfying a secondary air-in-line threshold different from the default air-in-line threshold, wherein the adjusted pressure threshold is different from the default pressure threshold; and
triggering the occlusion alarm based on the adjusted pressure threshold.

18. The non-transitory, computer-readable medium of claim 17, further comprising instructions that, when executed, cause the one or more processors to:
before triggering the occlusion alarm:
adjust the secondary air-in-line threshold based on the determined pressure decay; and
adjust the adjusted pressure threshold based on the detected amount of air.

19. The non-transitory, computer-readable medium of claim 17, further comprising instructions that, when executed, cause the one or more processors to:
before triggering the occlusion alarm:
adjust, based on a flow rate of the fluid inside the infusion line, at least one of the adjusted pressure threshold and the secondary air-in-line threshold.

20. The non-transitory, computer-readable medium of claim 17, wherein the adjusted pressure is set to a predetermined percentage of the default pressure threshold or the secondary air-in-line threshold is set to a predetermined percentage of the default air-in-line threshold.
